(19) United States
(12) Patent Application Publication
(10) Pub. No.: US 2011/0263442 A1
(43) Pub. Date: Oct. 27, 2011

(54) METHOD OF USING BIOMARKERS

(76) Inventor: Jan Akervall, Ann Arbor, MI (US)

(21) Appl. No.: 13/126,129

(22) PCT Filed: Oct. 22, 2009
(86) PCT No.: PCT/US09/61670

§ 371 Date: Jul. 15, 2011

Related U.S. Application Data
(60) Provisional application No. 61/109,331, filed on Oct. 29, 2008.

(51) Int. Cl. C40B 30/00; H01J 49/26; G01N 21/64; C12Q 1/68; G01N 33/566

(52) U.S. Cl. 506/7; 435/6.11; 435/7.21; 435/7.1; 436/501; 250/282

(57) ABSTRACT

The present invention provides methods and compositions for predicting patient responses to cancer treatment using the biomarkers: YAP-1, bcl-2, VEGF-c, c-met, and claudin-4.

### RELATED APPLICATION

This application claims the benefit of U.S. Provisional Application No. 61/109,331, filed Oct. 29, 2008, the entire contents of which are incorporated herein by reference.

### BACKGROUND

Primary surgical therapy for oropharyngeal and laryngeal SCHNN (squamous cell carcinoma of the head and neck) has given way to definitive radiotherapy or concurrent chemoradiotherapy as the sole modality or with chemotherapy in favor of organ preservation. To support this, randomized trials have proven the benefit of using radiation therapy (RT) for organ preservation in patients with squamous cell carcinoma of the head and neck and a reported local control benefit as well as improvement in survival with the delivery of chemotherapy concurrently with radiotherapy (CRT). Now, with the advent of recent imaging modalities such as PET/CT (positron emission tomography/computed tomography) there are fewer planned neck dissections in favor of observing the neck in patients with initial nodal positive disease that has a clinical response to CRT.

The above mentioned studies have provided the basis for current treatment decision which are primarily based on the patient's tumor-nodal-metastases (TNM) staging. However, patients with the same TNM stages have heterogeneous responses to therapy. Because these patients are often receiving RT and CRT as the sole therapy it becomes important to determine how each patient could respond to their respective therapy, and to separate those who are at high risk for local recurrence.

Due to the heterogeneous nature of tumors, it is less likely that any one specific marker will have prognostic or predictive value. Thus, there is a need in the art to identify biomarkers for use in assessing pre-treatment biopsies to predict the clinical response to RT and CRT in patients with head and neck cancer.

### SUMMARY

In one aspect, the present invention provides for methods for predicting the response to chemoradiotherapy treatment in a patient suffering from a head or neck cancer to comprising: measuring in a biological sample from said patient the protein or mRNA levels of (i) YAP-1, or (ii) at least one biomarker selected from (b) and at least one biomarker from (c): (b) bcl-2, and VEGF-c, (c) c-met, and claudin-4; or (iii) a combination of (i) and (ii). In certain embodiments, the mRNA levels are determined by measuring the levels of one or more nucleic acid sequences selected from the group consisting of: SEQ ID NO: 1 (YAP-1), SEQ ID NO: 2 (bcl-2); SEQ ID NO: 3 (VEGF-c); SEQ ID NO: 4 (c-met); and SEQ ID NO: 5 (claudin-4). In other embodiments, the protein levels are determined by measuring the levels of one or more amino acid sequences selected from the group consisting of: SEQ ID NO: 6 (YAP-1), SEQ ID NO: 7 (bcl-2); SEQ ID NO: 8 (VEGF-c); SEQ ID NO: 9 (c-met); and SEQ ID NO: 10 (claudin-4). In particular embodiments, the head or neck cancer is squamous cell carcinoma of the head and neck. In other embodiments, the cancer is oropharyngeal or laryngeal squamous cell carcinoma.

Further objects, features and advantages of this invention will become readily apparent to persons skilled in the art after a review of the following description, with reference to the drawings and claims that are appended to and form a part of this specification.

### BRIEF DESCRIPTION OF THE DRAWINGS

FIG. 1 depicts immunohistochemical staining of YAP-1 (A), bcl-2 (B), VEGF-c (C), c-met (D), claudin-4 (E) in biopsy specimens of patients with head and neck squamous cell carcinoma;

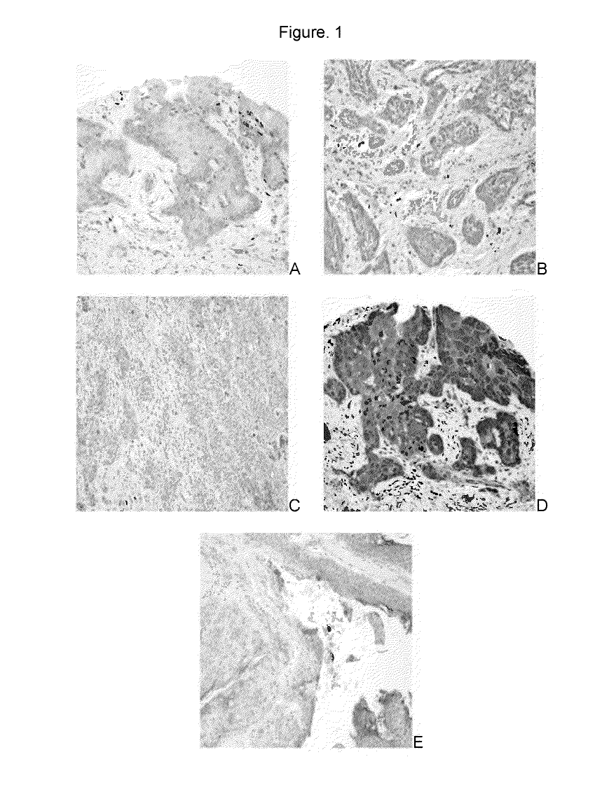

FIG. 2 depicts a Kaplan-Meier curve of the probability of recurrence free survival versus months for patients expressing high and low YAP-1, bcl-2, VEGF-c, c-met, and claudin-4;

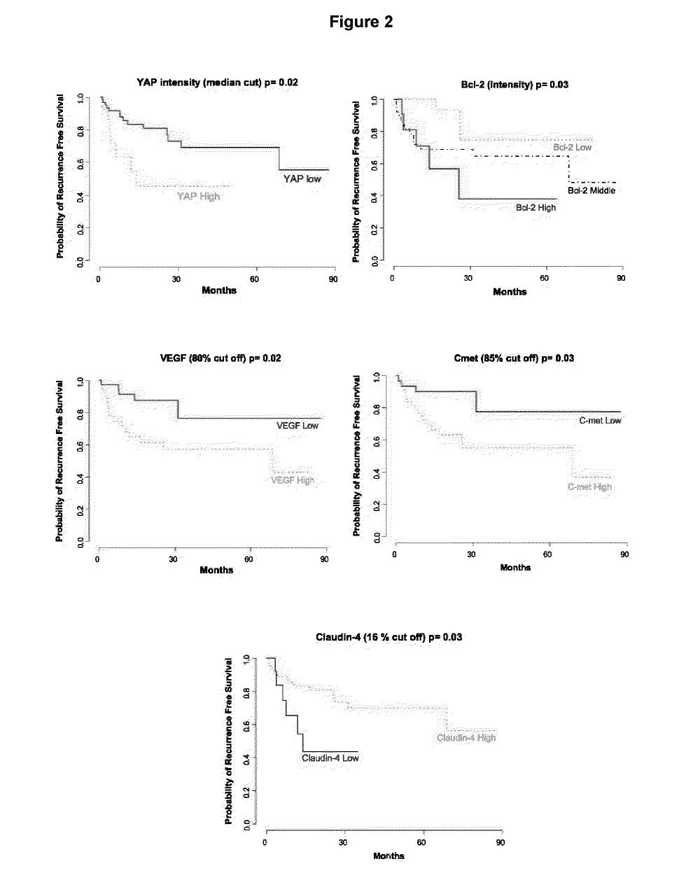

FIG. 3 depicts a Kaplan-Meier curve of the cause specific survival (%) versus months for patients expressing high and low levels of YAP-1, bcl-2, and VEGF-c.

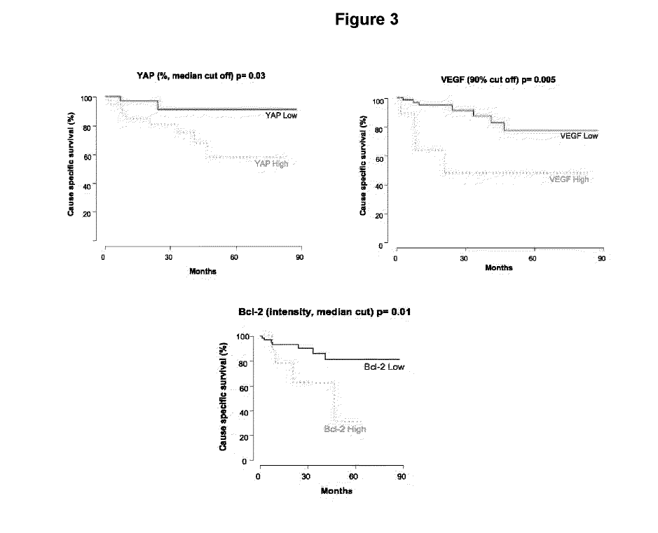

### DETAILED DESCRIPTION

The present invention provides methods and compositions for the use of biomarkers to predict the response to chemoradiotherapy of a head and neck cancer patient. In particular the present invention provides for methods for predicting the response to chemoradiotherapy treatment in a patient suffering from a head or neck cancer to comprising: measuring in a biological sample from said patient the protein or mRNA levels of (i) YAP-1, or (ii) at least one biomarker selected from (b) and at least one biomarker from (c): (b) bcl-2, and VEGF-c, (c) c-met, and claudin-4; or (iii) a combination of (i) and (ii).

In certain embodiments, the methods involve measuring in a biological sample from a patient the nucleic acid levels of one or more of YAP-1 (Yes-associated protein 65 kDa), bcl-2 (B-cell CLL/lymphoma 2), VEGF-c (vascular endothelial growth factor C), c-met, or claudin-4. Examples of nucleic acids associated with each of YAP-1, bcl-2, VEGF-c, c-met, or claudin-4 are in Table 1:

TABLE 1

| Biomarker | SEQ ID NO: | GenBank Accession No. |
|---|---|---|
| YAP-1 | SEQ ID NO: 1 | NM_006106 |
| bcl-2 | SEQ ID NO: 2 | M14745 |
| VEGF-c | SEQ ID NO: 3 | BC035212 |
| c-met | SEQ ID NO: 4 | NM_000245 |
| claudin-4 | SEQ ID NO: 5 | NM_001305 |

Examples of expressed sequence tag nucleic acid sequences that are associated with each of YAP-1, c-met, or claudin-4 are in Table 2:

TABLE 2

| Biomarker | SEQ ID NO: | GenBank Accession No. |
|---|---|---|
| YAP-1 | SEQ ID NO: 11 | AA708798 |
| c-met | SEQ ID NO: 12 | AA191433 |
| claudin-4 | SEQ ID NO: 13 | AA430665 |

In certain embodiments, the methods involve measuring in a biological sample from a patient the protein levels of one or more of YAP-1, bcl-2, VEGF-c, c-met, or claudin-4. Examples of amino acid sequences associated with each of YAP-1, bcl-2, VEGF-c, c-met, or claudin-4 are in Table 3:

TABLE 3

| Biomarker | SEQ ID NO: | GenBank Accession No. |
|---|---|---|
| YAP-1 | SEQ ID NO: 6 | NP_006097 |
| bcl-2 | SEQ ID NO: 7 | AAA35591 |
| VEGF-c | SEQ ID NO: 8 | AAH35212 |
| c-met | SEQ ID NO: 9 | NP_000236 |
| claudin-4 | SEQ ID NO: 10 | NP_001296 |

To examine the levels of mRNA or protein expression of one or more biomarkers, a biological sample of a head or neck cancer patient is typically assayed. A “biological sample” includes a sample from a tumor, a cancerous tissue, a pre-cancerous tissue, a biopsy, blood, serum, saliva, or a tissue, etc. obtained from a patient suffering from a head or neck cancer or who has yet to be diagnosed with a head or neck cancer.

The biological sample is then typically assayed from the presence of one or more expression products of a biomarker gene such as mRNA, cDNA, cRNA, protein, etc.

In one embodiment, a sample comprising RNA from a biological sample is used directly to measure the mRNA levels of a biomarker. In one particular embodiment, RNA is obtained from a biological sample. The RNA is then transformed into cDNA (complementary DNA) copy using methods known in the art. In particular embodiments, the cDNA is labeled with a fluorescent label or other detectable label. The cDNA is then hybridized to a substrate containing a one or more probes of interest. A probe of interest typically hybridizes under stringent hybridization conditions to the DNA sequence of interest. In certain embodiments, one or more nucleic acid probes are capable of hybridizing to the sequences of interest (e.g., any of SEQ ID NOS: 1-5, 11-13, or fragments thereof (e.g., fragments at least 15 nucleotides in length)) under the hybridization conditions of 6×SSC (0.9 M NaCl, 0.09 M sodium citrate, pH 7.4) at 65° C. The probes may comprise nucleic acids. An example of a nucleic acid is DNA. The term “nucleic acid” refers to deoxyribonucleotides or ribonucleotides and polymers thereof. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, and peptide-nucleic acids (PNAs).

In certain cases, the probes will be from about 15 to about 50 base pairs in length. The amount of cDNA hybridization can be measured by assaying for the presence of the detectable label, such as a fluorophore. The amount of the hybridization signal can be used to generate a qualitative or quantitative measurement of the level of a nucleic acid of interest in sample.

The term “detectable label” refers to a moiety that is attached through covalent or non-covalent means to an entity being measured or a probe. A “detectable label” can be a radioactive moiety, a fluorescent moiety, a chemiluminescent moiety, etc. The term “fluorescent label” refers to label that accepts radiant energy of one wavelength and emits radiant energy of a second wavelength. The presence of a detectable label may be assayed using methods known in the art that are appropriate to detect a particular label, such as spectrophotometric means (e.g., a spectrophotometer), radiometric means (e.g., scintillation counter), fluorometer, luminometer, etc.

Included within the scope of the invention are DNA microarrays containing a plurality of sequences that hybridize under stringent hybridization conditions to one or more biomarker gene sequences. An example of a substrate containing one or more probes of interest is a plurality of DNA probes that are affixed to a substrate. In certain embodiments, the substrate may comprise one or more materials such as gel, nitrocellulose, nylon, quartz, glass, metal, silica based materials, silica, resins, polymers, etc., or combinations thereof. Typically, the DNA probes comprise about 10-50 bp of contiguous DNA. In certain embodiments, the DNA probes are from about 20 to about 50 bp of contiguous DNA. In certain embodiments, the present invention relates to kits which comprise a microarray of one or more sequences capable of stringently hybridizing to any of SEQ ID NOS: 1-5, 11-13 or the complementary strand of any of SEQ ID NOS: 1-5, 11-13 and its directions for its use. The kit may comprise a container which comprises one or more microarrays and directions for their use.

The biological sample may also be analyzed for mRNA of one or more biomarkers using methods that can detect nucleic acids including, but not limited to, PCR (polymerase chain reaction); RT-PCR (reverse transcriptase-polymerase chain reaction); quantitative PCR, etc.

In certain embodiments, the levels of biomarker protein are measured by detecting the protein expression products of the genes or DNA sequences (e.g., any of SEQ ID NOS: 6-10). The levels of protein products may be measured using methods known in the art including the use of antibodies which specifically bind to a particular protein. These antibodies, including polyclonal or monoclonal antibodies, may be produced using methods that are known in the art. These antibodies may also be coupled to a solid substrate to form an antibody chip or antibody microarray. Antibody or protein microarrays may be made using methods that are known in the art. In addition, immunoassays, including immunohistochemistry, may be employed. In certain embodiments, the present invention relates to kits which comprise reagents (such as antibodies) capable of specifically binding to any of SEQ ID NOS: 6-10 and its directions for its use. The kit may comprise a container which comprises one or more reagents and directions for their use. Furthermore, mass spectrometry may be used to detect proteins or fragments thereof, and may be used in combination with other techniques such as HPLC.

The treatment of head and neck cancer in certain embodiments, involves measuring the levels of mRNA or protein of one or more biomarkers selected from the group consisting of YAP-1, bcl-2, VEGF-c, c-met, and claudin-4. The method of treatment typically further comprises administering a therapeutically effective amount of one or more cancer treatment agents selected from the group consisting of: cancer chemotherapeutic agents and radiation. The treatment of cancer may also comprise surgery or surgical procedures. The term “administering” refers to the method of contacting a compound with a subject. Modes of “administering” may include but are not limited to, methods that involve contacting the cancer chemotherapeutic agents intravenously, intraperitoneally, intranasally, transdermally, topically, via implantation, subcutaneously, parentally, intramuscularly, orally, systemically, and via adsorption. The term “treatment” includes the acute or prophylactic diminishment or alleviation of at least one symptom or characteristic associated or caused by the cancer being treated. For example, treatment can include diminishment of several symptoms of a cancer or complete eradication of a cancer. The phrase “therapeutically effective amount” means an amount of a cancer chemotherapeutic agent, or a pharmaceutically acceptable salt thereof, that is sufficient to inhibit, halt, or allow an improvement in the cancer being treated when administered alone or in conjunction with another pharmaceutical agent or treatment in a particular subject or subject population. For example in a human a therapeutically effective amount can be determined experimentally in a clinical setting, for the particular disease and subject being treated. It should be appreciated that determination of proper dosage forms, dosage amounts and routes of administration is within the level of ordinary skill in the pharmaceutical and medical arts.

It is within the purview of the skill medical practitioner to select an appropriate therapeutic regimen. Therapeutic regimens may be comprised of the use of cancer chemotherapeutic agents and/or radiation. Chemoradiotherapy is the use of both radiation and chemotherapy to treat a patient suffering from a cancer. The radiation and chemotherapy do not have to occur simultaneously and can be separated in time, for example by hours, days, or months, etc. A cancer chemotherapeutic agent is a chemical or biological agent (e.g., antibody, protein, RNA, DNA, etc.) that retards, slows, or stops the growth of cancer or is approved to treat a cancer by the U.S. Food and Drug Administration. Examples of head and neck cancer chemotherapeutic agents include, but are not limited to cisplatin, cetuximab, docetaxel, and erlotinib. In particular cases, the chemoradiotherapy comprises administering cisplatin and 5-fluorouracil. Another example of a cancer treatment agent is radiation. In certain embodiments, the cancer is a head or neck cancer. Examples of head or neck cancer include, but are not limited to: squamous cell carcinoma of the head and neck. Further examples of head and neck cancers include oropharyngeal and laryngeal squamous cell carcinoma.

### EXAMPLES

Purpose: To correlate expression of VEGF-c, bcl-2, claudin-4, c-met, and YAP-1 in pre-treatment biopsies with clinical outcomes in patients with squamous carcinoma of the head and neck (SCCHN).

Methods: From December 1995 to November 2004, 86 patients with clinical stage II-IVa SCCHN who underwent radiotherapy (RT) alone or concurrent cisplatinum based chemoradiotherapy (CRT) were selected. Immunohistochemical staining (IHC) for VEGF-c, bcl-2, claudin-4, YAP-1 and c-met proteins was performed on pretreatment biopsy specimens obtained from all 86 patients. Staining was graded according to intensity and percentage of cells positive by two independent observers.

Results: The median follow-up was 33.8 months. Twelve patients experienced an IR (incomplete response) and 11 patients recurred at a median time of 12.6 months. Of the recurrences, 7 patients were found to have a LRR (locoregional recurrence) and 4 patients were found to have distant metastases. Cause specific survival (CSS) and recurrence free survival (RFS) at 2 years was 85% and 90% and at 3 years was 81% and 84%, respectively. Biomarkers predictive for IR were increased VEGF-c (p=0.02), YAP-1(p<0.01), claudin-4 (p<0.01), c-met (p<0.01) and bcl-2 (p=0.02). Biomarkers predictive of RFS were YAP-1 (p=0.01) and bcl-2 (p<0.01). Biomarkers predictive of CSS were YAP-1 (p=0.04), VEGF-c (p=0.03), and claudin-4 (p=0.03).

Conclusion: All biomarkers were predictive of IR; in addition claudin-4 and VEGF-c predicted for CSS and bcl-2 for RFS. YAP-1 was the universal marker in predicting for all endpoints. Analyzing individual genetic profiles in the clinical setting using the above markers may allow for tailored patient-specific therapy to improve outcomes.

### Materials and Methods

Patients

One hundred and thirty one patients consecutively treated with primary RT or CRT for squamous cell carcinoma of the oropharynx and larynx from May 1995-July 2004 at William Beaumont Hospital were identified for this Human Investigation Committee approved study. Forty-five patients were excluded from the analysis due to previous history of carcinoma excluding non-melanomatous skin cancer, treatment received at an outside institution, unavailable treatment record or lack of tissue available for analysis. From December 1995 to November 2004, 86 patients with clinical stage II-IVa oropharyngeal (n=30) and stage I-IVa laryngeal (n=56) SCCHN who underwent treatment with either RT alone (n=47) or CRT (n=39) were selected for analysis.

Treatment

The primary site was treated with 6 mV photons. For early stage larynx patients, two-dimensional radiotherapy planning utilizing a 5×5 or 6×6 field box was used. Prior to 2004, most of the remaining patients were treated with three-dimensional conformal radiotherapy; thereafter, intensity modulated radiotherapy was used to maximally spare normal tissues. Fifteen patients received treatment twice daily, five days per week, with 120 cGy (centigray) per fraction. Seventy-one patients received treatment once daily, five days per week, with fraction dose ranging from 180-225 cGy per fraction. Median total dose to the primary site was 7000 cGy, (ranged from 2400 cGy-8160 cGy), and the median overall treatment time was 50 days (range, 16 to 69 days). One patient received 2400 cGy in a hypofractionated regimen secondary to difficulty with treatment compliance. All CRT patients received platinum based chemotherapy delivered concurrently with the radiotherapy. Treatment response was evaluated during and after treatment using nasopharyngoscopy, computed tomography, and/or biopsies. Any locoregional recurrence within 6 months after completion of radiation therapy was considered an incomplete response defined as either a partial response or no response.

Tumor Samples

Tumor samples were collected with the approval of the institutional human investigational committee from the eighty-six patients as described above. All of the tumors were fixed in formalin and embedded in paraffin wax. A head and neck pathologist reviewed the histopathology of each case by light microscopy of the hematoxylin and eosin stained sections and identified the appropriate regions of tissue for the immunohistochemistry to be performed.

Tissue Array

One to four 1.5 mm punch biopsies were taken from the paraffin embedded specimens after microscopic identification of representative tumor containing areas in hematoxylin/eosin (H&E) stained sections. The punch biopsies were then mounted on new paraffin blocks, yielding up to 100 specimens in each individual block. Five micrometer sections were then cut in a regular fashion for immunohistochemical analysis.

Immunohistochemistry

Immunohistochemical staining (IHC) for VEGF, bcl-2, claudin-4, YAP-1 and c-met was performed on pretreatment biopsy specimens obtained from all 86 patients. Tissue array technique was used to analyze areas of interest in each specimen. These areas were marked together with a pathologist. Hematoxylin/Eosin (H&E) was used to stain specific sections. To detect the five proteins, immunohistochemistry was completed using the Discovery XT System (Ventana, Tucson, Ariz.) at the following dilutions: bcl-2 (1:200), c-met (1:50), claudin-4 (1:50), VEGF-c (1:100) and YAP-1 (1:25). Antigen retrieval was performed using citrate buffer at pH 6 at 95° C. for 25 minutes. For quenching of endogenous peroxidases, sections were blocked with normal horse serum 2.5% (R.T.U. Vectastain Kit, Vector Laboratories, Burlingame, Calif.) for 20 minutes. A secondary antibody was added (R.T.U. Biotinylated Universal antibody Anti-rabbit/mouse IgG, Vectastain ABC-kit, Vector Laboratories, Burlingame, Calif.) for 30 minutes. After rinsing in PBS, specimens were incubated in ABC-reagent (Vectastain ABC-kit, Vector Laboratories, Burlingame, Calif.) for 30 minutes, and counterstained with hematoxylin.

The slides were then scored by two independent observers; staining was graded according to intensity and percentage of cells positive. If there was a discrepancy between the two independent observations, a third independent observer scored the slides and the results were averaged.

Statistical Analysis

Incomplete response was defined as either gross or microscopic persistent viable tumor after treatment or recurrence of tumor within six month after completion of treatment. Recurrence-free survival was defined as the time from radiotherapy completion to the first relapse (after six months from completion of treatment), death, or last follow-up, whichever occurred first. Cause specific survival was defined as the time from radiotherapy completion to death due to disease or last follow-up. Cumulative survival probabilities were plotted using Kaplan-Meier curves and compared with log-rank test. All tests were declared statistically significant if the calculated p value was ≦0.05. The chi-squared test was used to detect any correlation between biomarkers and clinical treatment factors. The statistical analysis was performed with Version 5.0 of the SAS statistical software package (SAS Institute Inc, Cary, N.C.) and (R version 2.6.1).

### Results

Patient/Tumor Characteristics

Median follow up was 29 months. The clinicopathologic characteristics are summarized in Table 4. As can be seen, the median age was 63 years (range, 40-95 years). Sixty-nine (80%) of 86 patients were male. Thirty-one patients had oropharyngeal primaries and 55 had laryngeal primaries. Of the laryngeal SCCHN, 41 patients had T1-T2 lesions, 14 patients had T3-T4 lesions, and 10 had locoregional lymph node involvement. Of the oropharyngeal SCCHN, 19 patients had T1-T2 lesions, 12 patients had T3-T4 lesions, and 22 patients had lymph node involvement.

TABLE 4
Patient Characteristics

| Factor | n (%) |
|---|---|
| Sex | |
| Male | 69 (80%) |
| Female | 17 (20%) |
| Subsite | |
| Oropharynx | 31 (36%) |
| Larynx | 55 (64%) |
| T stage | |
| T1-T2 | 60 (70%) |
| T3-T4 | 26 (30%) |
| Nodal Status | |
| N0 | 54 (63%) |
| N+ | 32 (37%) |
| AJCC Stage | |
| I | 20 (23%) |
| II | 18 (21%) |
| III | 20 (23%) |
| IV | 28 (33%) |
| Chemotherapy | |
| Yes | 39 (45%) |
| No | 47 (55%) |

Clinical Outcome

Patients were evaluated during and after treatment for response via clinical exam, nasopharyngoscopy, radiographic study, or biopsy. Twelve patients (7 larynx, 5 oropharynx) experienced an incomplete response; clinical and treatment characteristics for these patients are summarized in Table 5. No patient with an incomplete response or recurrence received less than 6600 cGy. The only significant clinical factor in the prediction of an incomplete response was having a radiation course time of greater than 50 days (p=0.04). There was an increased chance of experiencing distant failure with an incomplete response to therapy (p<0.01).

TABLE 5
Clinical and treatment characteristic of
patients with incomplete response

| | | Overall | | N |
|---|---|---|---|---|
| Patient | Site | Stage | T Stage | Stage |
| 1 | Larynx | 1 | 1a | 0 |
| 2 | Larynx | 1 | 1a | 0 |
| 3 | Larynx | 2 | 2 | 0 |
| 4 | Oropharynx | 2 | 2 | 0 |
| 5 | Oropharynx | 3 | 3 | 0 |
| 6 | Larynx | 3 | 3 | 0 |
| 7 | Larynx | 3 | 3 | 0 |
| 8 | Larynx | 3 | 3 | 0 |
| 9 | Larynx | 3 | 2 | 1 |
| 10 | Oropharynx | 4a | 1 | 2 |
| 11 | Oropharynx | 4a | 4 | 2a |
| 12 | Oropharynx | 4b | 4 | 3 |

Eleven patients (8 larynx, 3 oropharynx) were noted to have recurrence with a median time to recurrence of 12.5 months (range, 7.6-67.8 months). Of the recurrences, 7 patients were found to have a locoregional recurrence (median, 10.8 months) and 4 patients were found to have distant metastases (median, 25.2 months).

Recurrence free survival (RFS) and cause specific survival (CSS) at 2 years was 90% and 85% and at 3 years was 84% and 81%, respectively. Treatment variables analyzed including age, gender, primary site, clinical stage, primary treatment, radiation dose, and elapsed days during the radiation course. Univariate analysis revealed older age (p=0.04) and primary treatment (RT vs. CRT) (p=0.03) to predict for RFS. Primary treatment course was then analyzed in Stage<=2 (n=38) vs. Stage>2 (n=48) and node positive patients (n=54) vs. node negative patients (n=32). Chemoradiation has significant effect on Stage 3 and 4 patient group and Node positive patient groups, as expected. Older age when analyzed as a continuous variable (p=0.04) and age greater or less than 62 (p=0.03) as well as higher clinical T stage (0.03) predicted for CSS on UVA (univariate analysis).

Predictive Biomarkers

Biomarkers Predictive of Incomplete Response

Biomarkers predictive for incomplete response were increased VEGF-c optimal cut of 78.6% (p=0.02), YAP-1 intensity grading (p<0.01), YAP-1 intensity grading as a continuous variable (p<0.01), claudin-4 optimal cut of 85.1% (p<0.01), c-met intensity grading (p<0.01) and bcl-2 intensity grading (p=0.02).

Biomarkers Predictive of Recurrence Free Survival

Biomarkers predictive of RFS were YAP-1 optimal cut of 37.8% (p=0.01), YAP-1 intensity grading (p=0.03), and bcl-2 optimal cut of 10% (p<0.01).

Biomarkers Predictive of Cause Specific Survival

Biomarkers predictive of CSS were YAP-1 median intensity grading (p=0.04), YAP-1 optimal cut of 81.8% (p=0.02), VEGF-c optimal cut of 78.6% (p=0.03), and claudin-4 optimal cut of 85.1% (p=0.03).

Significant biomarkers were tested against significant clinical factors for each end point to test the independent prognostic significance of the biomarker. YAP-1 and bcl-2 were found to be independent of age at diagnosis in predicting for RFS, YAP-1 and VEGF-c were independent of age at diagnosis for CSS and VEGF-c was independent of Clinical T stage as a continuous variable for CSS. All biomarkers were independent of days of radiation in predicting for incomplete response. Interestingly, a log rank score was evaluated for both CSS and RFS and revealed a combination of clinical stage, claudin-4, YAP-1 and age to be very significant for CSS (p<0.01) and age, YAP-1, claudin-4 and primary treatment to be significant for RFS (p=0.02).

When the biomarkers YAP-1, bcl-2, VEGF-c, c-met, and claudin-4 were analyzed with regards to grade of positive cells and percentage of positive cells, the combination of the two, i.e. grade multiplied by percentage, had a synergistic effect. The combination of grade and percentage according to this formula improved the significance of the biomarkers when analyzed for response to therapy and prognosis. Furthermore, the combination of c-met and YAP-1 was a more significant predictor of response to radiation based therapy in the entire population (n=86), with 90% correct prediction of outcome of therapy, as compared to when these 2 markers were analyzed in subgroups consistent of either patients treated with radiation alone or in patients treated with chemoradiation.

Discussion

As primary treatment of SCCHN has shifted towards the use of radiotherapy or chemoradiotherapy, predicting tumor response to both modalities is useful in tailoring patient specific therapy. Treatment response to therapy was found to be independent of TNM stage in this study and patients with an incomplete response to their primary therapy (RT or CRT) were more likely to experience distant metastasis and have lower CSS than those with complete response. Therapy can be guided by evaluating individual tumor biology by assessing established prognostic and predictive biomarkers; this approach is already in use in early stage breast cancer to determine the benefit of chemotherapy.

The Yes Associated Protein (YAP-1) was found to be the universal biomarker in this study. The overexpression of YAP-1 has been seen in ependymoma (Modena et al. (2006)), NSCLC (non-small cell lung cancer) (Saviozzi et al. 2006), and pancreatic cancer (Guo et al. (2006)).

A previous study has shown that genes for the tyrosine kinase Yes-associated protein (YAP65) were preferentially expressed in transformed and metastatic tumor cell lines (Dong et al. (1997)). Silencing of YAP-1 reduces histone acetylation on the p53, resulting in delayed or reduced apoptosis mediated by p73 (Strano et al. (2005)).

Previous work has found low c-met expression to be associated with cisplatin sensitivity, and during the previous study concluded patients with tumors having high expression of c-met may not be good candidates for concomitant chemoradiation (Akervall et al. (2004)). This has been validated as in this study, c-met was significant in predicting for poor response to radiotherapy or chemoradiotherapy. MET is a tyrosine kinase receptor involved in proliferation, mitogenesis, angiogenesis, and metastasis. Overexpression of Met has been reported in breast, ovarian, thyroid, pancreatic, brain, and gastrointestinal tumors, and c-met overexpression has been correlated with poor prognosis in nasopharyngeal carcinoma patients (Qian et al. (2002)). Overexpression of c-met has also been shown to be a predictor of local recurrence in oral tongue carcinoma (Endo et al. (2006)). Currently, c-met was not predictive in CSS. There was no prediction of c-met in RFS; this may due to the fact that patients who had an incomplete response were censored in the RFS analysis.

In our study, Claudin-4 was found to be significant in predicting RFS. Claudin-4 encodes tight junction proteins. Its high expression has been noted in patients with breast cancer, urothelial, and prostate cancer. Increased claudin-4 in HNSCC and NSCLC cell lines were found to be associated with increases sensitivity to gefitinib (Frederick et al. (2007)), but this is targeted therapy and the conclusion may not be valid in this study. In serous papillary carcinoma, overexpression of claudin-4 was associated with poorer DFS and OS, with overexpression seen in the aggressive phenotyopes (Konecny et al. (2008)). In urothelial and prostate cancer it was found to be associated with stage and metastases. Perhaps, claudin-4 is a surrogate marker for tumor spread, which is why it may have lead to worse RFS.

In this study, bcl-2 was significant in predicting recurrence free survival and cause specific survival. Historically, it was discovered to be an anti apoptotic oncogene, suppressing p53. Most recently, in a cohort of nasopharynx patients who underwent high dose radiotherapy, bcl-2 overexpression had worse 5 year DFS (Chen et al. (2008)); 21 patients with locally advanced SCCHN who were treated with chemoradiation were found to have unfavorable outcome and shorter RFS if they overexpressed bcl-2 (Mannarini et al. (2007)).

Tumor hypoxia has been associated with poor response to radiation therapy; in this study VEGF-c was found to have significance in predicting RFS and CSS. VEGF-c is an angiogenesis promoting oncogene; previously, high expression of VEGF-c has been linked to poor response to radiation therapy. Pathologic complete response was assessed in patients undergoing preoperative rectal cancer; carcinomas that were considered VEGF-c positive were noted to have a pathological incomplete response (Zlobec et al. (2008)). In 27 patients with Stage II-IV SCCHN, high expression of VEGF-c significantly reduced local control and survival (Martin et al. (2007)). High levels of VEGF-c may be a surrogate for tumor hypoxia; hypoxia leads to radioresistance.

There are some limitations to this study. The patients that were analyzed were a heterogeneous group with a mixture of different head and neck disease sites and difference TNM stages. Patterns of spread to locoregional lymph nodes differences amongst patients with glottic primaries versus oropharyngeal primaries. Also, as this study spanned a time period of 9 years, different radiotherapy techniques and chemotherapy regimens were used. However, a heterogeneous population similar to one identified in this study is more likely representative of the population encountered in the wide range of clinical practices throughout the country; therefore, it makes the use of the panel of markers more universally applicable. Also, the biases associated with all retrospective studies have to be considered.

### CONCLUSION

In this study, the prognostic and predictive abilities of five markers with potential importance for chemosensitivity or radiosensitivity (VEGF-c, bcl-2, claudin-4, c-met, and YAP-1) based on previous c-DNA microarray studies (published and unpublished) were evaluated. By assessing pre-treatment biopsies, YAP-1 was found to be a universal marker in predicting for RT/CRT response, CSS, and RFS. Claudin-4 and VEGF-c predict for both CSS and RFS, and bcl-2 predicts for RT/CRT response and RFS. By using the above tested biomarkers patients at high risk for local recurrence can be identified early and appropriate therapy can be delivered upfront to optimize cancer cure and decrease morbidity from necessary salvage therapy in addition to primary therapy.

### REFERENCES

Akervall et al. “Genetic and expression profiles of squamous cell carcinoma of the head and neck correlate with cisplatin sensitivity and resistance in cell lines and patients.” Clin Cancer Res. (2004) 10(24):8204-8213.
Chen et al. “Prognostic impact of bcl-2 expression on advanced nasopharyngeal carcinoma.” Head Neck. (2008) 30(8):1052-1057
Dong, G. et al. “Genes differentially expressed with malignant transformation and metastatic tumor progression of murine squamous cell carcinoma.” J Cell Biochem. Suppl 28-29 (1997): 90-100.
Endo et al. “Prognostic value of cell motility activation factors in patients with tongue squamous cell carcinoma.” Hum Pathol. (2006) 37(8):1111-1116.
Frederick et al. “Epithelial to mesenchymal transition predicts gefitinib resistance in cell lines of head and neck squamous cell carcinoma and non-small cell lung carcinoma.” Mol Cancer Ther. (2007) 6(6):1683-1691.
Guo, J. et al. “Yes-associated protein (YAP65) in relation to Smad7 expression in human pancreatic ductal adenocarcinoma.” Int. J. Mol. Med. 17.5 (2006): 761-67.
Konecny et al. “Claudin-3 and claudin-4 expression in serous papillary, clear-cell, and endometrioid endometrial cancer.” Gynecol Oncol. (2008) 109(2):263-269.
Mannarini et al. “Markers of chemoradiation resistance in patients with locally advanced head and neck squamous cell carcinoma, treated by intra-arterial carboplatin and concurrent radiation.” Acta Otorhinolaryngol Ital. (2007) 27(4):173-180
Martin et al. “Vascular endothelial growth factor expression predicts outcome after primary radiotherapy for head and neck squamous cell cancer.” Clin Oncol (R Coll Radiol). (2007) 19(1):71-76
Modena, P. et al. “Identification of tumor-specific molecular signatures in intracranial ependymoma and association with clinical characteristics.” J. Clin. Oncol. 24.33 (2006): 5223-33.
Qian et al. “Met protein expression level correlates with survival in patients with late-stage nasopharyngeal carcinoma.” Cancer Res. (2002)62(2):589-596
Saviozzi, S. et al. “Selection of suitable reference genes for accurate normalization of gene expression profile studies in non-small cell lung cancer.” BMC. Cancer 6 (2006): 200.
Strano, S. et al. “The transcriptional coactivator Yes-associated protein drives p73 gene-target specificity in response to DNA Damage.” Mol. Cell 18.4 (2005): 447-59.
Zlobec et al. “Combined analysis of VEGF and EGFR predicts complete tumour response in rectal cancer treated with preoperative radiotherapy.” Br J. Cancer. (2008) 98(2):450-456.

What is claimed:

1. A method for predicting the response to chemoradiotherapy treatment in a patient suffering from a head or neck cancer to comprising:
measuring in a biological sample from said patient the protein or mRNA levels of:
(i) YAP-1; or
(ii) at least one biomarker selected from (b) and at least one biomarker from (c):
(b) bcl-2, and VEGF-c,
(c) c-met, and claudin-4; or
(iii) a combination of (i) and (ii).

2. The method of claim 1, wherein the mRNA levels are determined by measuring the levels of one or more nucleic acid sequences selected from the group consisting of:
SEQ ID NO: 1 (YAP-1),
SEQ ID NO: 2 (bcl-2);
SEQ ID NO: 3 (VEGF-c);
SEQ ID NO: 4 (c-met); and
SEQ ID NO: 5 (claudin-4).

3. The method of claim 1, wherein the protein levels are determined by measuring the levels of one or more amino acid sequences selected from the group consisting of:
SEQ ID NO: 6 (YAP-1),
SEQ ID NO: 7 (bcl-2);
SEQ ID NO: 8 (VEGF-c);
SEQ ID NO: 9 (c-met); and
SEQ ID NO: 10 (claudin-4).

4. The method of claim 1, wherein said head or neck cancer is squamous cell carcinoma of the head and neck.

5. The method of claim 4, wherein said cancer is oropharyngeal or laryngeal squamous cell carcinoma.

6. The method of claim 1, wherein said biological sample is from a tumor, a cancerous tissue, a pre-cancerous tissue, a biopsy, blood, serum, saliva, or a tissue.

7. The method of claim 1, wherein said chemoradiotherapy comprises administering one or more agents selected from the group consisting of: cisplatin, cetuximab, docetaxel, and erlotinib.

8. The method of claim 7, wherein said chemoradiotherapy comprises administering cisplatin and 5-fluorouracil.

9. The method of claim 3, wherein the determination of the protein levels is carried out using immunohistochemistry, an immunoassay, a protein assay, mass spectrometry, immunofluorescence, or a combination thereof.

10. The method of claim 1, wherein the response to chemoradiotherapy treatment is whether the patient will have an incomplete response to the chemotherapy.


SEQUENCE LISTING (13 sequences)

SEQ ID NO: 1 - DNA, 5128 bp, Homo sapiens
ccgagtgcctcgcagcccctcccgaggcgcagccgccagaccagtggagccggggcgcagggcgggggcggaggcgccggggcgggggatgcggggccgcggcgcagccccccggccctgagagcgaggacagcgccgcccggcccgcagccgtcgccgcttctccacctcggcccgtggagccggggcgtccgggcgtagccctcgctcgcctgggtcagggggtgcgcgtcgggggaggcagaagccatggatcccgggcagcagccgccgcctcaaccggccccccagggccaagggcagccgccttcgcagcccccgcaggggcagggcccgccgtccggacccgggcaaccggcacccgcggcgacccaggcggcgccgcaggcaccccccgccgggcatcagatcgtgcacgtccgcggggactcggagaccgacctggaggcgctcttcaacgccgtcatgaaccccaagacggccaacgtgccccagaccgtgcccatgaggctccggaagctgcccgactccttcttcaagccgccggagcccaaatcccactcccgacaggccagtactgatgcaggcactgcaggagccctgactccacagcatgttcgagctcattcctctccagcttctctgcagttgggagctgtttctcctgggacactgacccccactggagtagtctctggcccagcagctacacccacagctcagcatcttcgacagtcttcttttgagatacctgatgatgtacctctgccagcaggttgggagatggcaaagacatcttctggtcagagatacttcttaaatcacatcgatcagacaacaacatggcaggaccccaggaaggccatgctgtcccagatgaacgtcacagcccccaccagtccaccagtgcagcagaatatgatgaactcggcttcagccatgaaccagagaatcagtcagagtgctccagtgaaacagccaccacccctggctccccagagcccacagggaggcgtcatgggtggcagcaactccaaccagcagcaacagatgcgactgcagcaactgcagatggagaaggagaggctgcggctgaaacagcaagaactgcttcggcaggtgaggccacaggagttagccctgcgtagccagttaccaacactggagcaggatggtgggactcaaaatccagtgtcttctcccgggatgtctcaggaattgagaacaatgacgaccaatagctcagatcctttccttaacagtggcacctatcactctcgagatgagagtacagacagtggactaagcatgagcagctacagtgtccctcgaaccccagatgacttcctgaacagtgtggatgagatggatacaggtgatactatcaaccaaagcaccctgccctcacagcagaaccgtttcccagactaccttgaagccattcctgggacaaatgtggaccttggaacactggaaggagatggaatgaacatagaaggagaggagctgatgccaagtctgcaggaagctttgagttctgacatccttaatgacatggagtctgttttggctgccaccaagctagataaagaaagctttcttacatggttatagagccctcaggcagactgaattctaaatctgtgaaggatctaaggagacacatgcaccggaaatttccataagccagttgcagttttcaggctaatacagaaaaagatgaacaaacgtccagcaagatactttaatcctctattttgctcttccttgtccattgctgctgttaatgtattgctgacctctttcacagttggctctaaagaatcaaaagaaaaaaactttttatttcttttgctattaaaactactgttcattttgggggctgggggaagtgagcctgtttggatgatggatgccattccttttgcccagttaaatgttcaccaatcattttaactaaatactcagacttagaagtcagatgcttcatgtcacagcatttagtttgttcaacagttgtttcttcagcttcctttgtccagtggaaaaacatgatttactggtctgacaagccaaaaatgttatatctgatattaaatacttaatgctgatttgaagagatagctgaaaccaaggctgaagactgttttactttcagtattttcttttcctcctagtgctatcattagtcacataatgaccttgattttattttaggagcttataaggcatgagacaatttccatataaatatattaattattgccacatactctaatatagattttggtggataattttgtgggtgtgcattttgttctgttttgttgggttttttgttttttttgtttttggcagggtcggtgggggggttggttggttggttggttttgtcggaacctaggcaaatgaccatattagtgaatctgttaatagttgtagcttgggatggttattgtagttgttttggtaaaatcttcatttcctggttttttttaccaccttatttaaatctcgattatctgctctctcttttatatacatacacacacccaaacataacatttataatagtgtggtagtggaatgtatccttttttaggtttccctgctttccagttaatttttaaaatggtagcgctttgtatgcatttagaatacatgactagtagtttatatttcactggtagtttaaatctggttggggcagtctgcagatgtttgaagtagtttagtgttctagaaagagctattactgtggatagtgcctaggggagtgctccacgccctctgggcatacggtagatattatctgatgaattggaaaggagcaaaccagaaatggctttattttctcccttggactaatttttaagtctcgattggaattcagtgagtaggttcataatgtgcatgacagaaataagctttatagtggtttaccttcatttagctttggaagttttctttgccttagttttggaagtaaattctagtttgtagttctcatttgtaatgaacacattaacgactagattaaaatattgccttcaagattgttcttacttacaagacttgctcctacttctatgctgaaaattgaccctggatagaatactataaggttttgagttagctggaaaagtgatcagattaataaatgtatattggtagttgaatttagcaaagaaatagagataatcatgattatacctttatttttacaggaagagatgatgtaactagagtatgtgtctacaggagtaataatggtttccaaagagtattttttaaaggaacaaaacgagcatgaattaactcttcaatataagctatgaagtaatagttggttgtgaattaaagtggcaccagctagcacctctgtgttttaagggtctttcaatgtttctagaataagcccttattttcaagggttcataacaggcataaaatctcttctcctggcaaaagctgctatgaaaagcctcagcttgggaagatagatttttttccccccaattacaaaatctaagtattttggcccttcaatttggaggagggcaaaagttggaagtaagaagttttattttaagtactttcagtgctcaaaaaaatgcaatcactgtgttgtatataatagttcataggttgatcactcataataattgactctaaggcttttattaagaaaacagcagaaagattaaatcttgaattaagtctggggggaaatggccactgcagatggagttttagagtagtaatgaaattctacctagaatgcaaaattgggtatatgaattacatagcatgttgttgggattttttttaatgtgcagaagatcaaagctacttggaaggagtgcctataatttgccagtagccacagattaagattatatcttatatatcagcagattagctttagcttagggggagggtgggaaagtttggggggggggttgtgaagatttagggggaccttgatagagaactttataaacttctttctctttaataaagacttgtcttacaccgtgctgccattaaaggcagctgttctagagtttcagtcacctaagtacacccacaaaacaatatgaatatggagatcttcctttacccctcaactttaatttgcccagttatacctcagtgttgtagcagtactgtgatacctggcacagtgctttgatcttacgatgccctctgtactgacctgaaggagacctaagagtcctttccctttttgagtttgaatcatagccttgatgtggtctcttgttttatgtccttgttcctaatgtaaaagtgcttaactgcttcttggttgtattgggtagcattgggataagattttaactgggtattcttgaattgcttttacaataaaccaattttataatctttaaatttatcaactttttacatttgtgttattttcagtcagggcttcttagatctacttatggttgatggagcacattgatttggagtttcagatcttccaaagcactatttgttgtaataacttttctaaatgtagtgcctttaaaggaaaaatgaacacagggaagtgactttgctacaaataatgttgctgtgttaagtattcatattaaatacatgccttctatatggaacatggcagaaagactgaaaaataacagtaattaattgtgtaattcagaattcataccaatcagtgttgaaactcaaacattgcaaaagtgggtggcaatattcagtgcttaacacttttctagcgttggtacatctgagaaatgagtgctcaggtggattttatcctcgcaagcatgttgttataagaattgtgggtgtgcctatcataacaattgttttctgtatcttgaaaaagtattctccacattttaaatgttttatattagagaattctttaatgcacacttgtcaaatatatatatatagtaccaatgttacctttttattttttgttttagatgtaagagcatgctcatatgttaggtacttacataaattgttacattattttttcttatgtaatacctttttgtttgtttatgtggttcaaatatattctttccttaaaaaaaaaaaaaaaaaaaaaaaaaaaaaaaaaaaaaaa

SEQ ID NO: 2 - DNA, 6030 bp, Homo sapiens
gttggcccccgttacttttcctctgggaaatatggcgcacgctgggagaacagggtacgataaccgggagatagtgatgaagtacatccattataagctgtcgcagaggggctacgagtgggatgcgggagatgtgggcgccgcgcccccgggggccgcccccgcgccgggcatcttctcctcgcagcccgggcacacgccccatacagccgcatcccgggacccggtcgccaggacctcgccgctgcagaccccggctgcccccggcgccgccgcggggcctgcgctcagcccggtgccacctgtggtccacctgaccctccgccaggccggcgacgacttctcccgccgctaccgccgcgacttcgccgagatgtccaggcagctgcacctgacgcccttcaccgcgcggggacgctttgccacggtggtggaggagctcttcagggacggggtgaactgggggaggattgtggccttctttgagttcggtggggtcatgtgtgtggagagcgtcaaccgggagatgtcgcccctggtggacaacatcgccctgtggatgactgagtacctgaaccggcacctgcacacctggatccaggataacggaggctgggatgcctttgtggaactgtacggccccagcatgcggcctctgtttgatttctcctggctgtctctgaagactctgctcagtttggccctggtgggagcttgcatcaccctgggtgcctatctgggccacaagtgaagtcaacatgcctgccccaaacaaatatgcaaaaggttcactaaagcagtagaaataatatgcattgtcagtgatgttccatgaaacaaagctgcaggctgtttaagaaaaaataacacacatataaacatcacacacacagacagacacacacacacacaacaattaacagtcttcaggcaaaacgtcgaatcagctatttactgccaaagggaaatatcatttattttttacattattaagaaaaaaagatttatttatttaagacagtcccatcaaaactcctgtctttggaaatccgaccactaattgccaagcaccgcttcgtgtggctccacctggatgttctgtgcctgtaaacatagattcgctttccatgttgttggccggatcaccatctgaagagcagacggatggaaaaaggacctgatcattggggaagctggctttctggctgctggaggctggggagaaggtgttcattcacttgcatttctttgccctgggggctgtgatattaacagagggagggttcctgtggggggaagtccatgcctccctggcctgaagaagagactctttgcatatgactcacatgatgcatacctggtgggaggaaaagagttgggaacttcagatggacctagtacccactgagatttccacgccgaaggacagcgatgggaaaaatgcccttaaatcataggaaagtatttttttaagctaccaattgtgccgagaaaagcattttagcaatttatacaatatcatccagtaccttaagccctgattgtgtatattcatatattttggatacgcaccccccaactcccaatactggctctgtctgagtaagaaacagaatcctctggaacttgaggaagtgaacatttcggtgacttccgcatcaggaaggctagagttacccagagcatcaggccgccacaagtgcctgcttttaggagaccgaagtccgcagaacctgcctgtgtcccagcttggaggcctggtcctggaactgagccggggccctcactggcctcctccagggatgatcaacagggcagtgtggtctccgaatgtctggaagctgatggagctcagaattccactgtcaagaaagagcagtagaggggtgtggctgggcctgtcaccctggggccctccaggtaggcccgttttcacgtggagcatgggagccacgacccttcttaagacatgtatcactgtagagggaaggaacagaggccctgggcccttcctatcagaaggacatggtgaaggctgggaacgtgaggagaggcaatggccacggcccattttggctgtagcacatggcacgttggctgtgtggccttggcccacctgtgagtttaaagcaaggctttaaatgactttggagagggtcacaaatcctaaaagaagcattgaagtgaggtgtcatggattaattgacccctgtctatggaattacatgtaaaacattatcttgtcactgtagtttggttttatttgaaaacctgacaaaaaaaaagttccaggtgtggaatatgggggttatctgtacatcctggggcattaaaaaaaaaatcaatggtggggaactataaagaagtaacaaaagaagtgacatcttcagcaaataaactaggaaatttttttttcttccagtttagaatcagccttgaaacattgatggaataactctgtggcattattgcattatataccatttatctgtattaactttggaatgtactctgttcaatgtttaatgctgtggttgatatttcgaaagctgctttaaaaaaatacatgcatctcagcgtttttttgtttttaattgtatttagttatggcctatacactatttgtgagcaaaggtgatcgttttctgtttgagatttttatctcttgattcttcaaaagcattctgagaaggtgagataagccctgagtctcagctacctaagaaaaacctggatgtcactggccactgaggagctttgtttcaaccaagtcatgtgcatttccacgtcaacagaattgtttattgtgacagttatatctgttgtccctttgaccttgtttcttgaaggtttcctcgtccctgggcaattccgcatttaattcatggtattcaggattacatgcatgtttggttaaacccatgagattcattcagttaaaaatccagatggcaaatgaccagcagattcaaatctatggtggtttgacctttagagagttgctttacgtggcctgtttcaacacagacccacccagagccctcctgccctccttccgcgggggctttctcatggctgtccttcagggtcttcctgaaatgcagtggtgcttacgctccaccaagaaagcaggaaacctgtggtatgaagccagacctccccggcgggcctcagggaacagaatgatcagacctttgaatgattctaatttttaagcaaaatattattttatgaaaggtttacattgtcaaagtgatgaatatggaatatccaatcctgtgctgctatcctgccaaaatcattttaatggagtcagtttgcagtatgctccacgtggtaagatcctccaagctgctttagaagtaacaatgaagaacgtggacgcttttaatataaagcctgttttgtcttctgttgttgttcaaacgggattcacagagtatttgaaaaatgtatatatattaagaggtcacgggggctaattgctggctggctgccttttgctgtggggttttgttacctggttttaataacagtaaatgtgcccagcctcttggccccagaactgtacagtattgtggctgcacttgctctaagagtagttgatgttgcattttccttattgttaaaaacatgttagaagcaatgaatgtatataaaagcctcaactagtcatttttttctcctcttcttttttttcattatatctaattattttgcagttgggcaacagagaaccatccctattttgtattgaagagggattcacatctgcatcttaactgctctttatgaatgaaaaaacagtcctctgtatgtactcctctttacactggccagggtcagagttaaatagagtatatgcactttccaaattggggacaagggctctaaaaaaagccccaaaaggagaagaacatctgagaacctcctcggccctcccagtccctcgctgcacaaatactccgcaagagaggccagaatgacagctgacagggtctatggccatcgggtcgtctccgaagatttggcaggggcagaaaactctggcaggcttaagatttggaataaagtcacagaatcaaggaagcacctcaatttagttcaaacaagacgccaacattctctccacagctcacttacctctctgtgttcagatgtggccttccatttatatgtgatctttgttttattagtaaatgcttatcatctaaagatgtagctctggcccagtgggaaaaattaggaagtgattataaatcgagaggagttataataatcaagattaaatgtaaataatcagggcaatcccaacacatgtctagctttcacctccaggatctattgagtgaacagaattgcaaatagtctctatttgtaattgaacttatcctaaaacaaatagtttataaatgtgaacttaaactctaattaattccaactgtacttttaaggcagtggctgtttttagactttcttatcacttatagttagtaatgtacacctactctatcagagaaaaacaggaaaggctcgaaatacaagccattctaaggaaattagggagtcagttgaaattctattctgatcttattctgtggtgtcttttgcagcccagacaaatgtggttacacactttttaagaaatacaattctacattgtcaagcttatgaaggttccaatcagatctttattgttattcaatttggatctttcagggattttttttttaaattattatgggacaaaggacatttgttggaggggtgggagggaggaacaatttttaaatataaaacattcccaagtttggatcagggagttggaagttttcagaataaccagaactaagggtatgaaggacctgtattggggtcgatgtgatgcctctgcgaagaaccttgtgtgacaaatgagaaacattttgaagtttgtggtacgacctttagattccagagacatcagcatggctcaaagtgcagctccgtttggcagtgcaatggtataaatttcaagctggatatgtctaatgggtatttaaacaataaatgtgcagttttaactaacaggatatttaatgacaaccttctggttggtagggacatctgtttctaaatgtttattatgtacaatacagaaaaaaattttataaaattaagcaatgtgaaactgaattggagagtgataatacaagtcctttagtcttacccagtgaatcattctgttccatgtctttggacaaccatgaccttggacaatcatgaaatatgcatctcactggatgcaaagaaaatcagatggagcatgaatggtactgtaccggttcatctggactgccccagaaaaataacttcaagcaaacatcctatcaacaacaaggttgttctgcataccaagctgagcacagaagatgggaacactggtggaggatggaaaggctcgctcaatcaagaaaattctgagactattaataaataagactgtagtgtagatactgagtaaatccatgcacctaaaccttttggaaaatctgccgtgggccctccagatagctcatttcattaagtttttccctccaaggtagaatttgcaagagtgacagtggattgcatttcttttggggaagctttcttttggtggttttgtttattataccttcttaagttttcaaccaaggtttgcttttgttttgagttactggggttatttttgttttaaataaaaataagtgtacaataagtgtttttgtattgaaagcttttgttatcaagattttcatacttttaccttccatggctctttttaagattgatacttttaagaggtggctgatattctgcaacactgtacacataaaaaatacggtaaggatactttacatggttaaggtaaagtaagtctccagttggccaccattagctataatggcactttgtttgtgttgttggaaaaagtcacattgccattaaactttccttgtctgtctagttaatattgtgaagaaaaataaagtacagtgtgagatactg

SEQ ID NO: 3 - DNA, 1948 bp, Homo sapiens
gcggggagctcggatgtccggtttcctgtgaggcttttacctgacacccgccgcctttccccggcactggctgggagggcgccctgcaaagttgggaacgcggagccccggacccgctcccgccgcctccggctcgcccagggggggtcgccgggaggagcccgggggagagggaccaggaggggcccgcggcctcgcaggggcgcccgcgcccccacccctgcccccgccagcggaccggtcccccacccccggtccttccaccatgcacttgctgggcttcttctctgtggcgtgttctctgctcgccgctgcgctgctcccgggtcctcgcgaggcgcccgccgccgccgccgccttcgagtccggactcgacctctcggacgcggagcccgacgcgggcgaggccacggcttatgcaagcaaagatctggaggagcagttacggtctgtgtccagtgtagatgaactcatgactgtactctacccagaatattggaaaatgtacaagtgtcagctaaggaaaggaggctggcaacataacagagaacaggccaacctcaactcaaggacagaagagactataaaatttgctgcagcacattataatacagagatcttgaaaagtattgataatgagtggagaaagactcaatgcatgccacgggaggtgtgtatagatgtggggaaggagtttggagtcgcgacaaacaccttctttaaacctccatgtgtgtccgtctacagatgtgggggttgctgcaatagtgaggggctgcagtgcatgaacaccagcacgagctacctcagcaagacgttatttgaaattacagtgcctctctctcaaggccccaaaccagtaacaatcagttttgccaatcacacttcctgccgatgcatgtctaaactggatgtttacagacaagttcattccattattagacgttccctgccagcaacactaccacagtgtcaggcagcgaacaagacctgccccaccaattacatgtggaataatcacatctgcagatgcctggctcaggaagattttatgttttcctcggatgctggagatgactcaacagatggattccatgacatctgtggaccaaacaaggagctggatgaagagacctgtcagtgtgtctgcagagcggggcttcggcctgccagctgtggaccccacaaagaactagacagaaactcatgccagtgtgtctgtaaaaacaaactcttccccagccaatgtggggccaaccgagaatttgatgaaaacacatgccagtgtgtatgtaaaagaacctgccccagaaatcaacccctaaatcctggaaaatgtgcctgtgaatgtacagaaagtccacagaaatgcttgttaaaaggaaagaagttccaccaccaaacatgcagctgttacagacggccatgtacgaaccgccagaaggcttgtgagccaggattttcatatagtgaagaagtgtgtcgttgtgtcccttcatattggaaaagaccacaaatgagctaagattgtactgttttccagttcatcgattttctattatggaaaactgtgttgccacagtagaactgtctgtgaacagagagacccttgtgggtccatgctaacaaagacaaaagtctgtctttcctgaaccatgtggataactttacagaaatggactggagctcatctgcaaaaggcctcttgtaaagactggttttctgccaatgaccaaacagccaagattttcctcttgtgatttctttaaaagaatgactatataatttatttccactaaaaatattgtttctgcattcatttttatagcaacaacaattggtaaaactcactgtgatcaatatttttatatcatgcaaaatatgtttaaaataaaatgaaaattgtattataaaaaaaaaaaaaaaaaaaaaaaaaaaaaaaaaaaaa

SEQ ID NO: 4 - DNA, 6641 bp, Homo sapiens
gccctcgccgcccgcggcgccccgagcgctttgtgagcagatgcggagccgagtggagggcgcgagccagatgcggggcgacagctgacttgctgagaggaggcggggaggcgcggagcgcgcgtgtggtccttgcgccgctgacttctccactggttcctgggcaccgaaagataaacctctcataatgaaggcccccgctgtgcttgcacctggcatcctcgtgctcctgtttaccttggtgcagaggagcaatggggagtgtaaagaggcactagcaaagtccgagatgaatgtgaatatgaagtatcagcttcccaacttcaccgcggaaacacccatccagaatgtcattctacatgagcatcacattttccttggtgccactaactacatttatgttttaaatgaggaagaccttcagaaggttgctgagtacaagactgggcctgtgctggaacacccagattgtttcccatgtcaggactgcagcagcaaagccaatttatcaggaggtgtttggaaagataacatcaacatggctctagttgtcgacacctactatgatgatcaactcattagctgtggcagcgtcaacagagggacctgccagcgacatgtctttccccacaatcatactgctgacatacagtcggaggttcactgcatattctccccacagatagaagagcccagccagtgtcctgactgtgtggtgagcgccctgggagccaaagtcctttcatctgtaaaggaccggttcatcaacttctttgtaggcaataccataaattcttcttatttcccagatcatccattgcattcgatatcagtgagaaggctaaaggaaacgaaagatggttttatgtttttgacggaccagtcctacattgatgttttacctgagttcagagattcttaccccattaagtatgtccatgcctttgaaagcaacaattttatttacttcttgacggtccaaagggaaactctagatgctcagacttttcacacaagaataatcaggttctgttccataaactctggattgcattcctacatggaaatgcctctggagtgtattctcacagaaaagagaaaaaagagatccacaaagaaggaagtgtttaatatacttcaggctgcgtatgtcagcaagcctggggcccagcttgctagacaaataggagccagcctgaatgatgacattcttttcggggtgttcgcacaaagcaagccagattctgccgaaccaatggatcgatctgccatgtgtgcattccctatcaaatatgtcaacgacttcttcaacaagatcgtcaacaaaaacaatgtgagatgtctccagcatttttacggacccaatcatgagcactgctttaataggacacttctgagaaattcatcaggctgtgaagcgcgccgtgatgaatatcgaacagagtttaccacagctttgcagcgcgttgacttattcatgggtcaattcagcgaagtcctcttaacatctatatccaccttcattaaaggagacctcaccatagctaatcttgggacatcagagggtcgcttcatgcaggttgtggtttctcgatcaggaccatcaacccctcatgtgaattttctcctggactcccatccagtgtctccagaagtgattgtggagcatacattaaaccaaaatggctacacactggttatcactgggaagaagatcacgaagatcccattgaatggcttgggctgcagacatttccagtcctgcagtcaatgcctctctgccccaccctttgttcagtgtggctggtgccacgacaaatgtgtgcgatcggaggaatgcctgagcgggacatggactcaacagatctgtctgcctgcaatctacaaggttttcccaaatagtgcaccccttgaaggagggacaaggctgaccatatgtggctgggactttggatttcggaggaataataaatttgatttaaagaaaactagagttctccttggaaatgagagctgcaccttgactttaagtgagagcacgatgaatacattgaaatgcacagttggtcctgccatgaataagcatttcaatatgtccataattatttcaaatggccacgggacaacacaatacagtacattctcctatgtggatcctgtaataacaagtatttcgccgaaatacggtcctatggctggtggcactttacttactttaactggaaattacctaaacagtgggaattctagacacatttcaattggtggaaaaacatgtactttaaaaagtgtgtcaaacagtattcttgaatgttataccccagcccaaaccatttcaactgagtttgctgttaaattgaaaattgacttagccaaccgagagacaagcatcttcagttaccgtgaagatcccattgtctatgaaattcatccaaccaaatcttttattagtggtgggagcacaataacaggtgttgggaaaaacctgaattcagttagtgtcccgagaatggtcataaatgtgcatgaagcaggaaggaactttacagtggcatgtcaacatcgctctaattcagagataatctgttgtaccactccttccctgcaacagctgaatctgcaactccccctgaaaaccaaagcctttttcatgttagatgggatcctttccaaatactttgatctcatttatgtacataatcctgtgtttaagccttttgaaaagccagtgatgatctcaatgggcaatgaaaatgtactggaaattaagggaaatgatattgaccctgaagcagttaaaggtgaagtgttaaaagttggaaataagagctgtgagaatatacacttacattctgaagccgttttatgcacggtccccaatgacctgctgaaattgaacagcgagctaaatatagagtggaagcaagcaatttcttcaaccgtccttggaaaagtaatagttcaaccagatcagaatttcacaggattgattgctggtgttgtctcaatatcaacagcactgttattactacttgggtttttcctgtggctgaaaaagagaaagcaaattaaagatctgggcagtgaattagttcgctacgatgcaagagtacacactcctcatttggataggcttgtaagtgcccgaagtgtaagcccaactacagaaatggtttcaaatgaatctgtagactaccgagctacttttccagaagatcagtttcctaattcatctcagaacggttcatgccgacaagtgcagtatcctctgacagacatgtcccccatcctaactagtggggactctgatatatccagtccattactgcaaaatactgtccacattgacctcagtgctctaaatccagagctggtccaggcagtgcagcatgtagtgattgggcccagtagcctgattgtgcatttcaatgaagtcataggaagagggcattttggttgtgtatatcatgggactttgttggacaatgatggcaagaaaattcactgtgctgtgaaatccttgaacagaatcactgacataggagaagtttcccaatttctgaccgagggaatcatcatgaaagattttagtcatcccaatgtcctctcgctcctgggaatctgcctgcgaagtgaagggtctccgctggtggtcctaccatacatgaaacatggagatcttcgaaatttcattcgaaatgagactcataatccaactgtaaaagatcttattggctttggtcttcaagtagccaaaggcatgaaatatcttgcaagcaaaaagtttgtccacagagacttggctgcaagaaactgtatgctggatgaaaaattcacagtcaaggttgctgattttggtcttgccagagacatgtatgataaagaatactatagtgtacacaacaaaacaggtgcaaagctgccagtgaagtggatggctttggaaagtctgcaaactcaaaagtttaccaccaagtcagatgtgtggtcctttggcgtgctcctctgggagctgatgacaagaggagccccaccttatcctgacgtaaacacctttgatataactgtttacttgttgcaagggagaagactcctacaacccgaatactgcccagaccccttatatgaagtaatgctaaaatgctggcaccctaaagccgaaatgcgcccatccttttctgaactggtgtcccggatatcagcgatcttctctactttcattggggagcactatgtccatgtgaacgctacttatgtgaacgtaaaatgtgtcgctccgtatccttctctgttgtcatcagaagataacgctgatgatgaggtggacacacgaccagcctccttctgggagacatcatagtgctagtactatgtcaaagcaacagtccacactttgtccaatggttttttcactgcctgacctttaaaaggccatcgatattctttgctcttgccaaaattgcactattataggacttgtattgttatttaaattactggattctaaggaatttcttatctgacagagcatcagaaccagaggcttggtcccacaggccacggaccaatggcctgcagccgtgacaacactcctgtcatattggagtccaaaacttgaattctgggttgaattttttaaaaatcaggtaccacttgatttcatatgggaaattgaagcaggaaatattgagggcttcttgatcacagaaaactcagaagagatagtaatgctcaggacaggagcggcagccccagaacaggccactcatttagaattctagtgtttcaaaacacttttgtgtgttgtatggtcaataacatttttcattactgatggtgtcattcacccattaggtaaacattcccttttaaatgtttgtttgttttttgagacaggatctcactctgttgccagggctgtagtgcagtggtgtgatcatagctcactgcaacctccacctcccaggctcaagcctcccgaatagctgggactacaggcgcacaccaccatccccggctaatttttgtattttttgtagagacggggttttgccatgttgccaaggctggtttcaaactcctggactcaagaaatccacccacctcagcctcccaaagtgctaggattacaggcatgagccactgcgcccagcccttataaatttttgtatagacattcctttggttggaagaatatttataggcaatacagtcaaagtttcaaaatagcatcacacaaaacatgtttataaatgaacaggatgtaatgtacatagatgacattaagaaaatttgtatgaaataatttagtcatcatgaaatatttagttgtcatataaaaacccactgtttgagaatgatgctactctgatctaatgaatgtgaacatgtagatgttttgtgtgtatttttttaaatgaaaactcaaaataagacaagtaatttgttgataaatatttttaaagataactcagcatgtttgtaaagcaggatacattttactaaaaggttcattggttccaatcacagctcataggtagagcaaagaaagggtggatggattgaaaagattagcctctgtctcggtggcaggttcccacctcgcaagcaattggaaacaaaacttttggggagttttattttgcattagggtgtgttttatgttaagcaaaacatactttagaaacaaatgaaaaaggcaattgaaaatcccagctatttcacctagatggaatagccaccctgagcagaactttgtgatgcttcattctgtggaattttgtgcttgctactgtatagtgcatgtggtgtaggttactctaactggttttgtcgacgtaaacatttaaagtgttatattttttataaaaatgtttatttttaatgatatgagaaaaattttgttaggccacaaaaacactgcactgtgaacattttagaaaaggtatgtcagactgggattaatgacagcatgattttcaatgactgtaaattgcgataaggaaatgtactgattgccaatacaccccaccctcattacatcatcaggacttgaagccaagggttaacccagcaagctacaaagagggtgtgtcacactgaaactcaatagttgagtttggctgttgttgcaggaaaatgattataactaaaagctctctgatagtgcagagacttaccagaagacacaaggaattgtactgaagagctattacaatccaaatattgccgtttcataaatgtaataagtaatactaattcacagagtattgtaaatggtggatgacaaaagaaaatctgctctgtggaaagaaagaactgtctctaccagggtcaagagcatgaacgcatcaatagaaagaactcggggaaacatcccatcaacaggactacacacttgtatatacattcttgagaacactgcaatgtgaaaatcacgtttgctatttataaacttgtccttagattaatgtgtctggacagattgtgggagtaagtgattcttctaagaattagatacttgtcactgcctatacctgcagctgaactgaatggtacttcgtatgttaatagttgttctgataaatcatgcaattaaagtaaagtgatgcaacatcttgtaaaaaaaaaaaaaaaaaaaa

SEQ ID NO: 5 - DNA, 1846 bp, Homo sapiens
aaaagtgcctttgttggcctgggctcaggaatccagagaaactggtcaggaggaggccccagtgacaaaaacccctccctctgcccccgcccctctgccagagccatataactgctcaacctgtccccgagagagagtgccctggcagctgtcggctggaaggaactggtctgctcacacttgctggcttgcgcatcaggactggctttatctcctgactcacggtgcaaaggtgcactctgcgaacgttaagtccgtccccagcgcttggaatcctacggcccccacagccggatcccctcagccttccaggtcctcaactcccgtggacgctgaacaatggcctccatggggctacaggtaatgggcatcgcgctggccgtcctgggctggctggccgtcatgctgtgctgcgcgctgcccatgtggcgcgtgacggccttcatcggcagcaacattgtcacctcgcagaccatctgggagggcctatggatgaactgcgtggtgcagagcaccggccagatgcagtgcaaggtgtacgactcgctgctggcactgccgcaggacctgcaggcggcccgcgccctcgtcatcatcagcatcatcgtggctgctctgggcgtgctgctgtccgtggtggggggcaagtgtaccaactgcctggaggatgaaagcgccaaggccaagaccatgatcgtggcgggcgtggtgttcctgttggccggccttatggtgatagtgccggtgtcctggacggcccacaacatcatccaagacttctacaatccgctggtggcctccgggcagaagcgggagatgggtgcctcgctctacgtcggctgggccgcctccggcctgctgctccttggcggggggctgctttgctgcaactgtccaccccgcacagacaagccttactccgccaagtattctgctgcccgctctgctgctgccagcaactacgtgtaaggtgccacggctccactctgttcctctctgctttgttcttccctggactgagctcagcgcaggctgtgaccccaggagggccctgccacgggccactggctgctggggactggggactgggcagagactgagccaggcaggaaggcagcagccttcagcctctctggcccactcggacaacttcccaaggccgcctcctgctagcaagaacagagtccaccctcctctggatattggggagggacggaagtgacagggtgtggtggtggagtggggagctggcttctgctggccaggatagcttaaccctgactttgggatctgcctgcatcggcgttggccactgtccccatttacattttccccactctgtctgcctgcatctcctctgttccgggtaggccttgatatcacctctgggactgtgccttgctcaccgaaacccgcgcccaggagtatggctgaggccttgcccacccacctgcctgggaagtgcagagtggatggacgggtttagaggggaggggcgaaggtgctgtaaacaggtttgggcagtggtgggggagggggccagagaggcggctcaggttgcccagctctgtggcctcaggactctctgcctcacccgcttcagcccagggcccctggagactgatcccctctgagtcctctgccccttccaaggacactaatgagcctgggagggtggcagggaggaggggacagcttcacccttggaagtcctggggtttttcctcttccttctttgtggtttctgttttgtaatttaagaagagctattcatcactgtaattattattattttctacaataaatgggacctgtgcacaggaaaaaaaaaaaaaaaaaaaaaaaa

SEQ ID NO: 6 - protein, 454 aa, Homo sapiens
MDPGQQPPPQPAPQGQGQPPSQPPQGQGPPSGPGQPAPAATQAAPQAPPAGHQIVHVRGDSETDLEALFNAVMNPKTANVPQTVPMRLRKLPDSFFKPPEPKSHSRQASTDAGTAGALTPQHVRAHSSPASLQLGAVSPGTLTPTGVVSGPAATPTAQHLRQSSFEIPDDVPLPAGWEMAKTSSGQRYFLNHIDQTTTWQDPRKAMLSQMNVTAPTSPPVQQNMMNSASAMNQRISQSAPVKQPPPLAPQSPQGGVMGGSNSNQQQQMRLQQLQMEKERLRLKQQELLRQVRPQELALRSQLPTLEQDGGTQNPVSSPGMSQELRTMTTNSSDPFLNSGTYHSRDESTDSGLSMSSYSVPRTPDDFLNSVDEMDTGDTINQSTLPSQQNRFPDYLEAIPGTNVDLGTLEGDGMNIEGEELMPSLQEALSSDILNDMESVLAATKLDKESFLTWL

SEQ ID NO: 7 - protein, 239 aa, Homo sapiens
MAHAGRTGYDNREIVMKYIHYKLSQRGYEWDAGDVGAAPPGAAPAPGIFSSQPGHTPHTAASRDPVARTSPLQTPAAPGAAAGPALSPVPPVVHLTLRQAGDDFSRRYRRDFAEMSRQLHLTPFTARGRFATVVEELFRDGVNWGRIVAFFEFGGVMCVESVNREMSPLVDNIALWMTEYLNRHLHTWIQDNGGWDAFVELYGPSMRPLFDFSWLSLKTLLSLALVGACITLGAYLGHK

SEQ ID NO: 8 - protein, 419 aa, Homo sapiens
MHLLGFFSVACSLLAAALLPGPREAPAAAAAFESGLDLSDAEPDAGEATAYASKDLEEQLRSVSSVDELMTVLYPEYWKMYKCQLRKGGWQHNREQANLNSRTEETIKFAAAHYNTEILKSIDNEWRKTQCMPREVCIDVGKEFGVATNTFFKPPCVSVYRCGGCCNSEGLQCMNTSTSYLSKTLFEITVPLSQGPKPVTISFANHTSCRCMSKLDVYRQVHSIIRRSLPATLPQCQAANKTCPTNYMWNNHICRCLAQEDFMFSSDAGDDSTDGFHDICGPNKELDEETCQCVCRAGLRPASCGPHKELDRNSCQCVCKNKLFPSQCGANREFDENTCQCVCKRTCPRNQPLNPGKCACECTESPQKCLLKGKKFHHQTCSCYRRPCTNRQKACEPGFSYSEEVCRCVPSYWKRPQMS

SEQ ID NO: 9 - protein, 1390 aa, Homo sapiens
MKAPAVLAPGILVLLFTLVQRSNGECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTGLIAGVVSISTALLLLLGFFLWLKKRKQIKDLGSELVRYDARVHTPHLDRLVSARSVSPTTEMVSNESVDYRATFPEDQFPNSSQNGSCRQVQYPLTDMSPILTSGDSDISSPLLQNTVHIDLSALNPELVQAVQHVVIGPSSLIVHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDTRPASFWETS

SEQ ID NO: 10 - protein, 209 aa, Homo sapiens
MASMGLQVMGIALAVLGWLAVMLCCALPMWRVTAFIGSNIVTSQTIWEGLWMNCVVQSTGQMQCKVYDSLLALPQDLQAARALVIISIIVAALGVLLSVVGGKCTNCLEDESAKAKTMIVAGVVFLLAGLMVIVPVSWTAHNIIQDFYNPLVASGQKREMGASLYVGWAASGLLLLGGGLLCCNCPPRTDKPYSAKYSAARSAAASNYV

SEQ ID NO: 11 - DNA, 472 bp, Homo sapiens
ttttttgtggcaataattaatatatttatatggaaattgtctcatgccttataagctcctaaaataaaatcaaggtcattatgtgactaatgatagcactaggaggaaaagaaaatactgaaagtaaaacagtcttcagccttggtttcagctatctcttcaaatcagcattaagtatttaatatcagatataacatttttggcttgtcagaccagtaaatcatgtttttccactggacaaaggaagctgaagaaacaactgttgaacaaactaaatgctgtgacatgaagcatctgacttctaagtctgagtatttagttaaaatgattggtgaacatttaactgggcaaaaggaatggcatccatcatccaaacaggctcacttcccccagcccccaaaatgaacagtagttttagatagcaaagaaataaaaagtttttttcttttgattgtttagagccaactgtgaa

SEQ ID NO: 12 - DNA, 504 bp, Homo sapiens
  misc_feature (468)..(468): n is a, c, g, or t
tttcaaactcctggactcaagaaatccacccacctcagcctcccaaagtgctaggattacaggcatgagccactgcgcccagcccttataaatttttgtatagacattcctttggttggaagaatatttataggcaatacagtcaaagtttcaaaatagcatcacacaaaacatgtttataaatgaacaggatgtaatgtacatagatgacattaagaaaatttgtatgaaataatttagtcatcatgaaatatttagttgtcatataaaaacccactgtttgagaatgatgctactctgatctaatgaatgtgaacatgtagatgttttgtgtgtatttttttaaatgaaaactcaaaataagacaagtaatttgttgataaatatttttaaagataactcagcatgtttgtaaagcaggataccattttacctaaaaggttcattggttccaatccacagctcatnggtagagcaaggaaggggtgganggatggaaaggat

SEQ ID NO: 13 - DNA, 242 bp, Homo sapiens
tttttcctgtgcacaggtcccatttattgtagaaaataataataattacagtgatgaatagctcttcttaaattacaaaacagaaaccacaaagaaggaagaggaaaaaccccaggacttccaagggtgaagctgtcccctcctccctgccaccctcccaggctcattagtgtccttggaaggggcagaggactcagaggggatcagtctccaggggccctgggctgaagcgggtgaggcag